(19) United States
(12) Patent Application Publication
(10) Pub. No.: US 2021/0321895 A1
(43) Pub. Date: Oct. 21, 2021

(54) INTEGRATED PRESSURE MONITORING SYSTEM FOR DETECTION OF VENTRICULOPERITONEAL (VP) SHUNT CLOGS

(71) Applicants: Rahul Ramanathan, Plainsboro, NJ (US); Noah Freedman, New Haven, CT (US); Eliza Schally, Cranberry Township, PA (US); Benjamin Rarrick, Clark's Summit, PA (US); Zhiwei Xie, San Ramon, CA (US)

(72) Inventors: Rahul Ramanathan, Plainsboro, NJ (US); Noah Freedman, New Haven, CT (US); Eliza Schally, Cranberry Township, PA (US); Benjamin Rarrick, Clark's Summit, PA (US); Zhiwei Xie, San Ramon, CA (US)

(21) Appl. No.: 17/234,978

(22) Filed: Apr. 20, 2021

Related U.S. Application Data
(60) Provisional application No. 63/012,411, filed on Apr. 20, 2020.

(51) Int. Cl. A61B 5/03; A61B 5/00

(52) U.S. Cl. CPC A61B 5/031; A61B 5/036; A61B 5/6852; A61B 2562/0247; A61B 5/6803; A61B 5/746; A61B 5/0004

(57) ABSTRACT

A pressure monitoring system is disclosed herein comprising at least one pressure and/or flow sensors that is fully integrated into a proximal catheter of a ventriculoperitoneal (VP) shunt. The pressure and/or flow sensor is wired to a transceiver, such as an inductively-charged Bluetooth wireless transceiver, on an external surface of a patient's skull to transmit a detected intracranial pressure (ICP) to a remote monitoring device, wherein the remote monitoring device comprises a computer and/or a mobile device with a compatible application for information analysis.

### CROSS-REFERENCE TO RELATED APPLICATIONS

The present application claims priority under 35 U.S.C. § 119(e) to U.S. Provisional Patent Application Ser. No. 63/012,411, titled INTEGRATED PRESSURE MONITORING SYSTEM FOR DETECTION OF VENTRICULOPERITONEAL (VP) SHUNT CLOGS, filed Apr. 20, 2020, the disclosure of which is herein incorporated by reference in its entirety.

### BACKGROUND

Hydrocephalus is a condition in which an accumulation of cerebrospinal fluid (CSF) occurs within the brain. Patients afflicted with hydrocephalus have excessive accumulation of CSF in their ventricles that cannot be naturally drained. There are several forms of the disease, including congenital hydrocephalus, which occurs in approximately 1 out of 1000 infants. Congenital hydrocephalus is treated by surgically inserting a ventriculoperitoneal (VP) shunt into a patient's ventricles to divert CSF to the peritoneum where the CSF can be absorbed. In such instances, the VP shunt is worn chronically as hydrocephalus is currently incurable. VP shunts frequently become clogged due to, at least in part, a buildup of biological residue in the shunt tubing. There is currently an absence of a non-invasive, cheap, and/or convenient manner to ascertain the presence of a clog.

A clog in the shunt causes intracranial pressure (ICP) to rise, the first symptoms of which include headache, nausea, and/or lightheadedness. When left unaddressed, a rise in ICP can lead to permanent brain damage. Currently, patients are required to see a medical professional upon exhibiting any of the early symptoms to ascertain the possibility of a clog. However, it is possible that a patient can present with one or more of these early symptoms independently of a VP shunt clog. For example, a patient may not have a VP shunt clog, but may have a head cold or some other ailment.

The general nature of the early symptoms requires a doctor to perform invasive neurosurgery to assess whether the patient's ICP is actually rising. In some cases, the shunt is not actually clogged and the surgery was unnecessary. Such instances waste time and money while also exposing the patient to an excessive, unnecessary risk. A pressure monitoring system, compatible with currently available VP shunts, is disclosed herein that is configured to measure ICP while alleviating the need for invasive neurosurgery.

### SUMMARY

The pressure monitoring system disclosed herein comprises at least one pressure and/or flow sensors that is fully integrated into a proximal catheter of a ventriculoperitoneal (VP) shunt. The pressure and/or flow sensor is wired to a transceiver, such as an inductively-charged Bluetooth wireless transceiver, on an external surface of a patient's skull to transmit a detected intracranial pressure (ICP) to a remote monitoring device. In various instances, the remote monitoring device comprises a computer and/or a mobile device with a compatible application for information analysis.

### BRIEF DESCRIPTION OF THE FIGURES

Various features of the embodiments described herein are set forth with particularity in the appended claims. The various embodiments, however, both as to organization and methods of operation, together with advantages thereof, may be understood in accordance with the following description taken in conjunction with the accompanying drawings as follows:

FIG. 1 is a diagram of a common cerebral shunt according to at least one aspect of the disclosure;

FIG. 2 is a perspective view of a pressure sensor for use with a proximal catheter pressure sensor according to at least one aspect of the disclosure;

FIG. 3 is a perspective view of a helix arrangement of a pressure monitoring system according to at least one aspect of the disclosure;

FIG. 4 is a perspective view of a heat-shrunk arrangement of a pressure monitoring system according to at least one aspect of the disclosure;

FIG. 5 is a Bluetooth® RF transceiver for use with a pressure monitoring system according to at least one aspect of the disclosure;

FIG. 6 is a perspective view of Bluetooth® enabled circuitry for use with a pressure monitoring system according to at least one aspect of the disclosure; and

FIG. 7 is a schematic of a pressure monitoring system according to at least one aspect of the disclosure.

Corresponding reference characters indicate corresponding parts throughout the several views. The exemplifications set out herein illustrate various embodiments of the invention, in one form, and such exemplifications are not to be construed as limiting the scope of the invention in any manner.

### DESCRIPTION

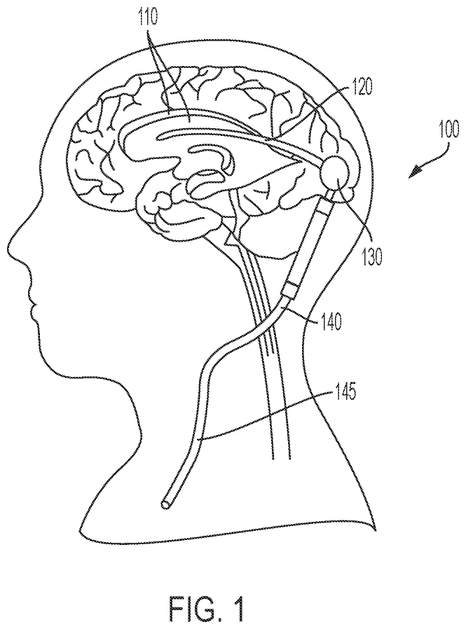

A pressure monitoring system configured to monitor an intracranial pressure (ICP) of a patient without invasive neurosurgery is disclosed herein. In patients who suffer from hydrocephalus or other related diseases, an accumulation of cerebrospinal fluid (CSF) occurs with the brain. Such an accumulation of CSF leads to an increase in pressure inside the skull, or intracranial pressure (ICP). In most cases, hydrocephalus is treated by the surgical placement of a shunt system. A shunt is a hollow tube surgically placed in a patient's brain to help drain cerebrospinal fluid and redirect it to another location in the body where it can be reabsorbed. Cerebrospinal fluid can be redirected to a patient's chest or abdominal cavity, for example. Shunts can come in a variety of forms; however, most shunts comprise a valve housing connected to a catheter, the end of which is typically placed in the patient's peritoneal cavity. A catheter is a flexible tube inserted through a narrow opening into a body cavity. As shown in FIG. 1, a common cerebral shunt 100 comprises a first, proximal catheter 120 located in a patient's ventricles 110. The location of the shunt can vary and is determined by a clinician based on the type and/or location of a blockage causing hydrocephalus, for example. The shunt 100 further comprises a valve 130 configured to open when the pressure in the brain gets too high. The valve 130 connects the first catheter 120 to a second catheter 140. A distal portion 145 of the second catheter 140 leads to another location in the patient's body, such as the chest or abdominal cavity, for example.

A leading cause of shunt failure includes, for example, the presence of an obstruction and/or a blockage of the shunt at either the proximal and/or distal ends. The shunt can become blocked due to the buildup of excess protein in a patient's cerebral spinal fluid, for example. Extra protein can collect at a point of drainage and slowly accumulate to clog the valve. Other causes of shunt blockage include, for example, overdrainage and/or slit ventricle syndrome.

The disclosed pressure monitoring system is small-profile, has minimal interaction with its surrounding environment, and has the ability to transmit detected information about pressure and/or flow wirelessly to a receiver outside the patient's body. In various instances, the receiver is positioned on an exterior surface of a patient's skin. A portion of the pressure monitoring system disclosed herein is implanted underneath the patient's skin in conjunction with proximal catheter architecture. While such a portion of the pressure monitoring system is envisioned to be implanted during placement of a VP shunt, the pressure monitoring system can be implanted after a VP shunt has already been placed. The implanted portion is configured to be stored beneath a patient's scalp for an extended duration of time, such that clinicians and/or patients can continue to interrogate and/or monitor the patient's intracranial pressure non-invasively when desired. Prior to being implanted, the implanted portion is encapsulated in a durable and/or flexible biocompatible material, such as an epoxy, for example.

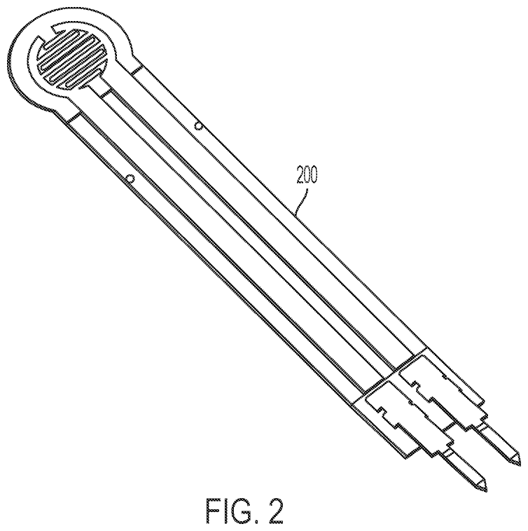

A pressure monitoring system comprises at least one pressure and/or flow sensor, a microcontroller comprising a transceiver, and a remote monitoring device. An example of a pressure sensor 200 suitable for use with the pressure monitoring system is shown in FIG. 2. The pressure and/or flow sensor is configured to detect a patient's intracranial pressure. The pressure and/or flow sensor is configured to be fully integrated with a proximal catheter, such as first catheter 120, of the VP shunt. In at least one instance, the pressure sensing subunit is integrated into a 3 mm diameter proximal catheter as a 5 mm diameter piece with multiple recording sites. These dimensions are intended to closely mimic those of modern VP shunt tubes. Detachment of the pressure sensor 200 from the catheter architecture can be prevented by ensuring that an adequate amount of adhesive and/or bonding material was used during attachment and/or encapsulating the pressure sensor and the catheter architecture in a durable and/or flexible material.

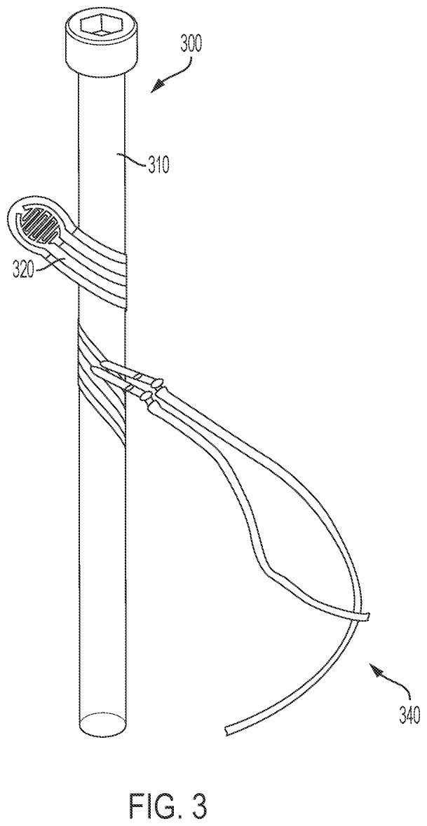

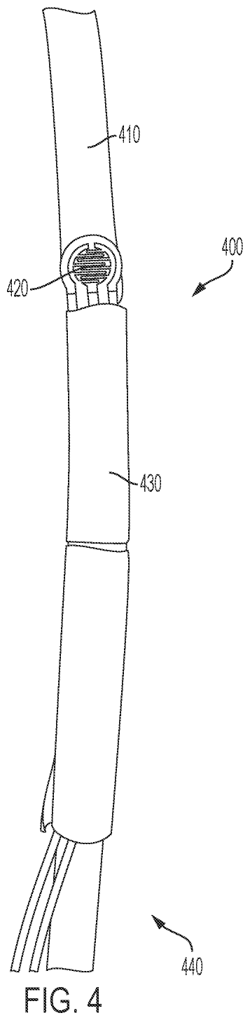

The sensor can be placed in any suitable location along the VP shunt. Multiple wiring configurations are envisioned, with a first arrangement involving a helix-formation configuration 300 as shown in FIG. 3. In the helix formation 300, a sensor 320 is wrapped and/or coiled around a first, or proximal, catheter 310 of the pressure monitoring system. The helix formation 300 is configured to reduce an overall profile of the VP shunt to minimize physical interactions with the environment. Using the helix-formation design 300, attachment between the main pressure-sensing region and the plastic tube via epoxy can be difficult to achieve. Modifying this region can also result in malfunctions in readings. FIG. 4 depicts an alternative embodiment 400, where a slimmer profile of the overall VP shunt is achieved by positioning a sensor 420 parallel to a first, or proximal, catheter 410. The sensor 420 was then heat-shrunk for attachment to the catheter 410 using a biocompatible and resilient material 430.

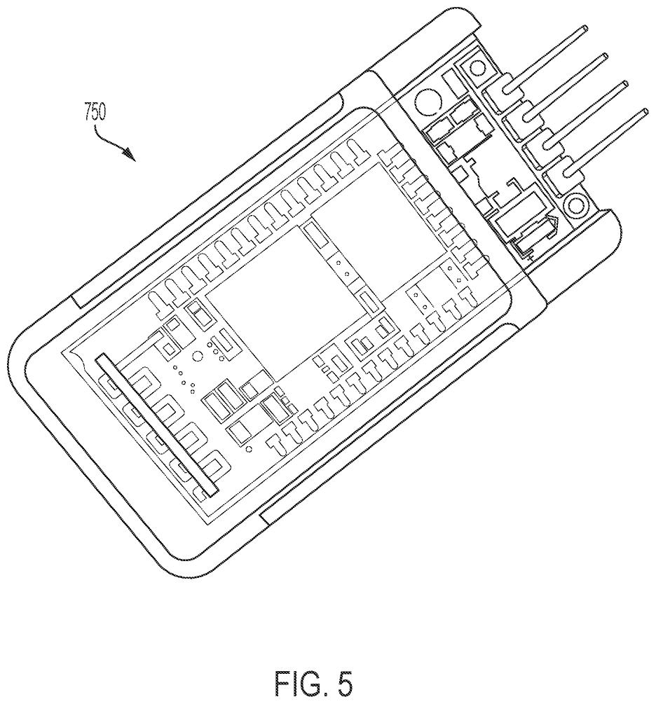

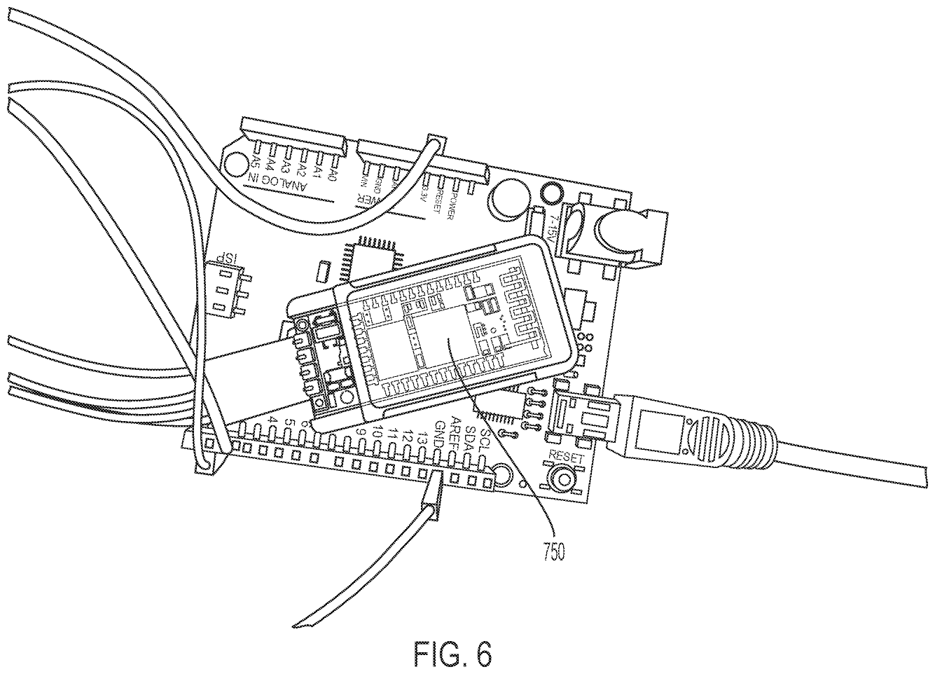

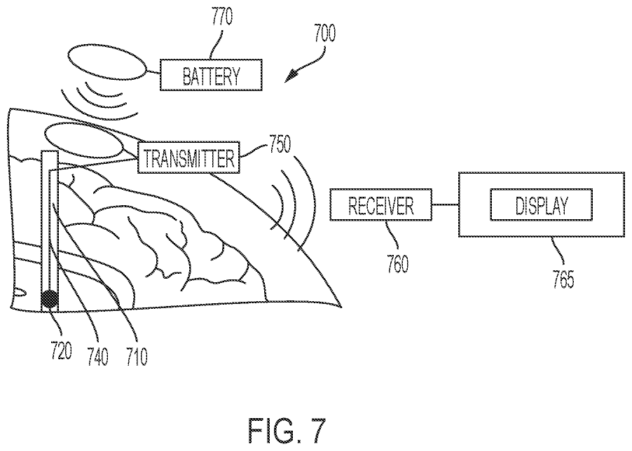

The microcontroller is intended to be positioned outside of a patient's skull, while remaining implanted underneath the patient's scalp. An exemplary pressure monitoring system 700 is depicted in FIG. 7. A sensor 720, such as a pressure and/or flow sensor, is attached to a first catheter 710 using any of the methods described herein or any suitable method. A wired connection 740, 340, 440 exists between the sensor 720 and a microcontroller 750; however, any suitable connection, such as a wireless connection, is envisioned. The microcontroller 750 comprises a transceiver configured to transmit the detected intracranial pressure to a receiver, or a remote monitoring device 760. An example of a suitable transceiver 750 is a Bluetooth® wireless transceiver, as shown in FIGS. 5 and 6. As discussed in greater detail below, the remote monitoring device 760 comprises a display 765 configured to display a detected parameter relating to a patient's current intracranial pressure. In various instances, the remote monitoring device 760 comprises a computer, a cell phone, a tablet, and/or any suitable device.

The pressure monitoring system 700 can be powered via induction from an external power source 770, without which the implanted circuit is passive. Shown in FIG. 6, the printed circuit components of the system are made from standard copper conductive tracks place onto CCL-Copper Clad Laminate. These parts are all internal to the pressure monitoring system. Stated another way, these parts are all intended to be implanted underneath patient's skin. In various instances, the inductive charging coil is made from copper, and contains a small circuitry component that is also CCL-Copper Clad Laminate. One coil is internal to the system and one coil is external to the system and the user's body. The implanted components are positioned in an epoxy casing to protect the electrical components from bodily fluids. In various instances, the epoxy casing is made from Bisphenol A epoxy resin. In various instances, the read out device is made from Acrylonitrile Butadiene Styrene (ABS) Filament.

In various instances, the inductive charging coil located outside of the patient is incorporated into a headband. In a clinical setting, nurses and/or physicians are able to easily put the headband comprising the inductive charging coil onto a patient's head. It is envisioned that a patient may have such a headband and/or inductive charging coil in the patient's residence. The detected pressure reading is then output to a provided output device, such as the remote monitoring device. The provided output device is configured to display a detected parameter relating to a patient's current ICP thereon in any suitable manner, as discussed in greater detail herein. The user can then read the detected parameter from the output device.

According to various embodiments, the remote monitoring device comprises a computer, a mobile device, a cell phone, and/or any device comprising compatible programming to receive and/or analyze the detected intracranial pressure information. The remote monitoring device comprises a processor and a memory. In various instances, a threshold value indicative of a threshold intracranial pressure is stored in the memory. The memory comprises readable and writable functionalities. The microprocessor and the read out device runs C++ code; however, any suitable coding language is envisioned. A default value can be stored in the memory indicative of an average threshold intracranial pressure. A clinician can adjust the stored threshold intracranial pressure value based on factors such as patient demographics, for example, as a desirable threshold intracranial pressure may be different from patient to patient and/or during the stages of a particular patient's treatment.

In at least one instance, the measured ICP is within +/−2 mmHg of the true value. The pressure sensor operates in an on-demand mode to measure the ICP in real-time when desired, such as when the patient is visiting the neurosurgeon, for example. In at least one instance, the inductive charger is able to power the internal circuitry from a distance of 1 cm. In at least one instance, the internal circuitry requires 20 mA to properly function. As discussed in greater detail herein, data transmission can be achieved using Bluetooth® technologies operating in the 2.402 GHz to 2,480 GHz range; however, any suitable communication means is envisioned. In at least one instance, the remote monitoring device is able to receive the ICP information and/or detected value from anywhere within 20 ft of the person with the internal circuitry; however, other communication means are envisioned where the remote monitoring device is able to receive the ICP value from an extended distance.

In various instances, the remote monitoring device is a computer affiliated with a health care provider. In such instances, the health care provider can be immediately notified if a particular patient's detected intracranial pressure exceeds a threshold value. In various instances, the remote monitoring device is a patient's personal mobile device. In such instances, the patient can go to a hospital to be observed by a clinician. In various instances, the patient's personal mobile device can alert a paramedic and/or hospital if the patient's detected intracranial pressure exceeds a threshold value. In various instances, the remote monitoring device is configured to alert a user when the detected intracranial pressure exceeds a threshold and/or when an error is detected. Such a detected error may indicate a problem with the power supply, for example. In various instances, the communication to the remote monitoring device may alert the user and/or clinician that the internal pressure monitoring system is recommended to be replaced due to length of implantation, damage, and/or exposure to excessive temperatures, for example.

Such an alert can be communicated through various forms of feedback, including, for example, haptic, acoustic, and/or visual feedback. In at least one instance, the feedback comprises audio feedback, and the remote monitoring device can comprise a speaker which emits a sound, such as a beep, for example, when a threshold is approached and/or exceeded and/or when an error is detected. In certain instances, the feedback comprises visual feedback and the remote monitoring device can comprise a light emitting diode (LED), for example, which flashes when a threshold is approached and/or exceeded and/or when an error is detected. In various instances, the feedback comprises haptic feedback and the remote monitoring device can comprise an electric motor comprising an eccentric element which vibrates when a threshold is approached and/or exceeded and/or when an error is detected. The alert can be specific or generic. For example, the alert can specifically state that the detected intracranial pressure is a particular value, or the alert can specifically state that the implanted sensor system has malfunctioned and/or is unable to detect an intracranial pressure.

A number of exemplary embodiments have been described. Nevertheless, it will be understood by one of ordinary skill in the art that various modifications may be made without departing from the spirit and scope of the techniques described herein.

While several forms have been illustrated and described, it is not the intention of the applicant to restrict or limit the scope of the appended claims to such detail. Numerous modifications, variations, changes, substitutions, combinations, and equivalents to those forms may be implemented and will occur to those skilled in the art without departing from the scope of the present disclosure. Moreover, the structure of each element associated with the described forms can be alternatively described as a means for providing the function performed by the element. Also, where materials are disclosed for certain components, other materials may be used. It is therefore to be understood that the foregoing description and the appended claims are intended to cover all such modifications, combinations, and variations as falling within the scope of the disclosed forms. The appended claims are intended to cover all such modifications, variations, changes, substitutions, modifications, and equivalents.

Any patent application, patent, non-patent publication, or other disclosure material referred to in this specification and/or listed in any Application Data Sheet is incorporated by reference herein, to the extent that the incorporated materials is not inconsistent herewith. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

In summary, numerous benefits have been described which result from employing the concepts described herein. The foregoing description of the one or more forms has been presented for purposes of illustration and description. It is not intended to be exhaustive or limiting to the precise form disclosed. Modifications or variations are possible in light of the above teachings. The one or more forms were chosen and described in order to illustrate principles and practical application to thereby enable one of ordinary skill in the art to utilize the various forms and with various modifications as are suited to the particular use contemplated. It is intended that the claims submitted herewith define the overall scope.

What is claimed is:

1. A pressure monitoring system, comprising:
a ventriculoperitoneal shunt, comprising:
a first catheter;
a second catheter; and
a valve connecting the first catheter to the second catheter;
a pressure sensor replaceably attached to the first catheter, wherein the pressure sensor is configured to detect a parameter relating to a current intracranial pressure;
a transceiver; and
a remote monitoring device configured to receive the detected parameter from the transceiver.

2. The pressure monitoring system of claim 1, further comprising an inductive charging coil.

3. The pressure monitoring system of claim 2, wherein the inductive charging coil is positioned on a removable headband worn by a user.

4. The pressure monitoring system of claim 1, wherein the remote monitoring device comprises a processor and a memory, and wherein a threshold pressure value is stored in the memory.

5. The pressure monitoring system of claim 4, wherein the remote monitoring device is configured to populate an alert when the threshold pressure value is exceeded.

6. The pressure monitoring system of claim 5, wherein the alert comprises a warning presented on the remote monitoring device.

7. The pressure monitoring system of claim 4, wherein the threshold pressure value is uniquely modifiable based on an identity of a user.

8. The pressure monitoring system of claim 1, wherein the pressure sensor is replaceably attached to the first catheter in a helical configuration.

9. The pressure monitoring system of claim 1, wherein the pressure sensor is replaceably attached parallel to the first catheter.

10. The pressure monitoring system of claim 1, wherein the transceiver wirelessly communicates the detected parameter to the remote monitoring device.

11. The pressure monitoring system of claim 1, further comprising a wired connection between the pressure sensor and the transceiver.

12. The pressure monitoring system of claim 1, wherein the pressure sensor is configured to detect a parameter at various points along a length of the first catheter.

13. The pressure monitoring system of claim 1, wherein the pressure sensor is encapsulated in a flexible biocompatible material.

14. A pressure monitoring system for use with a shunt, wherein the pressure monitoring system comprises:
a sensing circuit replaceably mounted to the shunt;
a transceiver; and
a remote monitoring device configured to receive the detected parameter from the transceiver.

15. The pressure monitoring system of claim 14, wherein the sensing circuit comprises a flow sensor.

16. The pressure monitoring system of claim 14, wherein the sensing circuit comprises a pressure sensor.

17. The pressure monitoring system of claim 14, wherein the sensing circuit is configured to detect a parameter at various points along a length of the shunt.

18. The pressure monitoring system of claim 14, wherein the remote monitoring device comprises a processor and a memory, and wherein a threshold pressure value is stored in the memory.

19. The pressure monitoring system of claim 18, wherein the remote monitoring device is configured to populate an alert when the threshold pressure value is exceeded.

20. A pressure monitoring system, comprising:
a shunt, comprising:
a first catheter;
a second catheter; and
a valve connecting the first catheter to the second catheter;
a sensor replaceably attached to the first catheter, wherein the sensor is configured to detect a parameter relating to a current intracranial pressure; and
a transceiver configured to communicate the detected parameter to a remote monitoring device.
